(19) United States
(12) Patent Application Publication
(10) Pub. No.: US 2002/0127587 A1
(43) Pub. Date: Sep. 12, 2002

(54) Methods and compositions for isolation of biological macromolecules

(76) Inventors: Domenica Simms, Silver Spring, MD (US); Thuan Trinh, Gaithersburg, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 10/073,260

(22) Filed: Feb. 13, 2002

Related U.S. Application Data
(60) Provisional application No. 60/268,027, filed on Feb. 13, 2001.

(51) Int. Cl. C12Q 1/68; C12N 1/08

(52) U.S. Cl. 435/6; 435/270

(57) ABSTRACT

The present invention relates generally to compositions, methods and kits for use in clarification and viscosity reduction of biological samples. More specifically, the invention relates to such compositions, methods and kits that are useful in the isolation of biological macromolecules from cells (e.g., bacterial cells, animal cells, fungal cells, viruses, yeast cells or plant cells) via lysis and one or more additional isolation procedures, such as one or more filtration procedures. In particular, the invention relates to compositions, methods and kits wherein biological macromolecules are isolated using a filter, where the pore size increases in the direction of sample flow. The compositions, methods and kits of the invention are suitable for isolating a variety of forms of biological macromolecules from cells. The compositions, methods and kits of the invention are particularly well-suited for rapid isolation of nucleic acid molecules from bacterial cells.

### CROSS REFERENCES TO RELATED APPLICATIONS

This application claims the benefit of priority to U.S. Provisional Application 60/268,027; filed Feb. 13, 2001, the contents of which are fully incorporated by reference herein.

### BACKGROUND OF THE INVENTION

1. Field of the Invention

The present invention is in the fields of molecular biology, biochemistry and genetics. The invention relates generally to compositions, methods and kits for use in viscosity reduction and clarification of biological samples. More specifically, the invention relates to such compositions, methods and kits that are useful in the isolation of biological macromolecules, particularly nucleic acid molecules. The compositions, methods and kits of the invention are suitable for treatment of biological samples, and isolation of biological macromolecules from a number of sources.

2. Background Art

Most methods for the isolation of biological macromolecules from biological samples utilize a lysing process that releases intracellular contents, including genomic DNA, into the surrounding solvent. The presence of this genomic DNA causes the lysate to be extremely viscous, compromising the subsequent yield and purity of the isolated biological macromolecules. The viscosity of the lysate is routinely reduced by shearing the genomic DNA, often via homogenization, which is routinely accomplished by forcing the lysate through a 20-gauge needle attached to a syringe. Alternatively, samples may be simultaneously lysed and homogenized by processing with a rotor-stator homogenizer. However, the use of a needle to homogenize samples is unsafe for the operator, and use of a rotor-stator homogenizer can lead to sample to sample contamination when processing multiple samples. Devices are available that shear genomic DNA by passing the lysate through tandem layers of polyethylene or polypropylene with progressively smaller pore sizes in the direction of flow (EP0616638B1), but particulates are not retained, and the clarified homogenate must be separated from the pellet of cell debris after centrifugation.

### BRIEF SUMMARY OF THE INVENTION

The present invention relates generally to compositions, methods and kits for use in shearing genomic DNA, and clarifying biological samples. More specifically, the invention relates to such compositions, methods and kits that aid in the isolation of biological macromolecules from cells (e.g., bacterial cells, animal cells, fungal cells, yeast cells or plant cells) and tissues via lysis and one or more additional isolation procedures, such as one or more filtration procedures. In particular, the invention relates to compositions, methods and kits for use in isolating biological macromolecules, particularly nucleic acid molecules, from a cellular lysate by passing the sample through a filter comprising one or more filters, wherein the pore size of the filters increase in the direction of sample flow. In another aspect of the invention, the filter of the invention comprises a first set or population of pores which are of sufficient size to retard the flow of cellular debris; and a second set or population of pores larger than the first set or population of pores. In a particularly preferred aspect of the invention, the filter of the invention comprises a first set or population of pores which are of sufficient size to retard the flow of cellular debris, and a second set or population of pores of sufficient size to shear genomic DNA.

Additionally, the invention relates to a method for isolation of biological macromolecules, said method comprising contacting a filter of the invention with a biological sample comprising the biological macromolecules of interest, wherein the pore size of said filter increases in the direction of sample flow.

More particularly, the invention relates to a method for isolation of biological macromolecules, by:

(a) contacting host cells containing the macromolecules of interest with a composition capable of lysing all or substantially all of said cells to give a lysate; and

(b) contacting the lysate with a filter (preferably the filter comprises two or more filters), and wherein the pore size increases in the direction of sample flow; and

(c) promoting the flow of the sample through the filter.

The invention also relates to isolated biological macromolecules produced by the methods of the invention. Additionally, the invention relates to host cells comprising these isolated nucleic acid molecules of the invention. The invention also relates to further manipulation (e.g. cloning, hybridization, restriction, etc.) of the isolated biological macromolecules; specifically further manipulation of isolated nucleic acid molecules of the invention.

In a related aspect, the invention relates to compositions for use in isolating the biological macromolecules of interest. The invention also relates to compositions made by the methods of the invention. Such compositions of the invention preferably comprise one or more components selected from the group consisting of one or more filters of the invention, one or more lysing agents, one or more buffers, one or more cells or tissues, one or more biological macromolecules, one or more solvents (organic or inorganic), and one or more alcohols. In a preferred embodiment, the composition of the invention comprises one or more filters, wherein the pore size of the filters increase in the direction of sample flow (FIG. 8).

In another preferred embodiment the filter of the invention comprises:

(a) a first filter, having pores of sufficient size to retard the flow of cellular debris; and

(b) a second filter, downstream of the first filter, having pores larger than those of said first filter.

The invention also relates to kits for use in isolating biological macromolecules of interest, comprising the filter of the invention. In related aspects, the kits of the invention further comprise one or more additional reagents, such as one or more lysis compositions, one or more restriction enzymes, one or more polypeptides having nucleic acid polymerase activity (e.g., one or more DNA polymerases which may be thermostable DNA polymerases and/or one or more reverse transcriptases which may be substantially reduced in RNase H activity), one or more cells competent for transformation (e.g., competent cells), transformation reagents, transfection reagents (e.g., cationic lipids) or other components or reagents that may be useful in conjunction with further purification, processing and analysis of the isolated macromolecules of the invention. Examples include, but are not limited to, components or reagents useful in nucleic acid purification, precipitation, hybridization, amplification, sequencing, cloning, transfection, transcription, translation, and the like. Such kits of the invention may also comprise protocols or instructions for carrying out the methods of the invention.

Other preferred embodiments of the present invention will be apparent to one of ordinary skill in light of what is known in the art, the following drawings and description of the invention, and the claims.

### BRIEF DESCRIPTION OF THE DRAWINGS/FIGURES

FIG. 1 is a graph representing the yield of RNA isolated from HeLa cells using a method of the invention.

FIG. 2 is a bar graph representing the yield of RNA isolated from rat liver using a precipitation based method.

FIG. 3 is a bar graph representing the yield of RNA isolated from rat liver using a method of the invention.

FIG. 4 is a graph representing the yield of RNA isolated from three different rat tissues (brain, liver and spleen) using a method of the invention.

FIG. 5 is a graph representing the yield of RNA isolated from sugarbeet leaves using a method of the invention.

FIG. 6 is a graph representing the yield of RNA isolated from human whole blood using a method of the invention.

FIG. 7 is a graph representing the yield of RNA isolated from yeast cells using a method of the invention.

FIGS. 8A and 8B is a schematic representation of a filter of the invention.

### DETAILED DESCRIPTION OF THE INVENTION

The present invention relates generally to compositions, methods and kits for use in shearing genomic DNA, and clarifying biological samples. More specifically, the invention relates to such compositions, methods and kits that aid in the isolation of biological macromolecules from cells (e.g., bacterial cells, animal cells, fungal cells, yeast cells or plant cells) via lysis and one or more additional isolation procedures, such as one or more filtration procedures. In particular, the invention relates to compositions, methods and kits for use in isolating biological macromolecules, particularly nucleic acid molecules, from a cellular lysate by passing the sample through a filter, where the pore size of the filter increases in the direction of sample flow through the filter.

In the description that follows, a number of terms used in the fields of molecular biology and recombinant DNA technology are utilized extensively. In order to provide a clearer and consistent understanding of the specification and claims, including the scope to be given such terms, the following definitions are provided.

Biological Macromolecule.

Any molecule contained in a biological source with a molecular weight greater than 250 daltons. The most common examples of biological macromolecules are polymeric in nature, and include DNA, RNA, derivatives of DNA and RNA, chimeric DNA/RNA molecules, proteins, peptides, or combinations thereof.

The methods of the invention are particularly well-suited for isolation of extrachromosomal nucleic acid molecules, including but not limited to RNA, mRNA, tRNA, plasmids, vectors, phagemids, cosmids, BACs, PACs, YACs, cDNA molecules or cDNA libraries, mitochondrial nucleic acid molecules, and chloroplast nucleic acid molecules, any of which may be single-stranded or double-stranded, linear or circular, supercoiled, and which may be DNA or RNA molecules.

Host.

Any prokaryotic or eukaryotic cell that is the recipient of a replicable expression vector or cloning vector. The terms “host” or “host cell” may be used interchangeably herein. For examples of such hosts, see Maniatis et al., “Molecular Cloning: A Laboratory Manual,” Cold Spring Harbor Laboratory, Cold Spring Harbor, N.Y. (1982). Preferred prokaryotic hosts include, but are not limited to, bacteria of the genus Escherichia (e.g., E. coli), Bacillus, Staphylococcus, Agrobacter (e.g., A. tumefaciens), Streptomyces, Pseudomonas, Salmonella, Serratia, Caryophanon, etc. The most preferred prokaryotic host is E. coli. Bacterial hosts of particular interest in the present invention include E. coli strains K12, DH10B, DH5α and HB101. Preferred eukaryotic hosts include, but are not limited to, fungi, fish cells, yeast cells, plant cells and animal cells. Particularly preferred animal cells are insect cells such as Drosophila cells, Spodoptera Sf9, Sf21 cells and Trichoplusa High-Five cells; nematode cells such as C. elegans cells; and mammalian cells such as COS cells, CHO cells, VERO cells, 293 cells, PERC6 cells, BHK cells and human cells. In accordance with the invention, a host or host cell may serve as the cellular source for the desired macromolecule to be isolated.

Vector.

A vector is a nucleic acid molecule (preferably DNA) capable of replicating autonomously in a host cell. Such vectors may also be characterized by having a small number of endonuclease restriction sites at which such sequences may be cut without loss of an essential biological function and into which nucleic acid molecules may be spliced to bring about its replication and cloning. Examples include plasmids, autonomously replicating sequences (ARS), centromeres, cosmids and phagemids. Vectors can further provide primer sites, e.g., for PCR, transcriptional and/or translational initiation and/or regulation sites, recombinational signals, recombination sites, replicons, etc. The vector can further contain one or more selectable markers suitable for use in the identification of cells transformed or transfected with the vector, such as kanamycin, tetracycline, amplicillin, etc.

In accordance with the invention, any vector may be used. In particular, vectors known in the art and those commercially available (and variants or derivatives thereof) may be used in accordance with the invention. Such vectors may be obtained from, for example, Vector Laboratories Inc., Invitrogen Corporation, Promega, Novagen, NEB, Clontech, Boehringer Mannheim, Pharmacia, EpiCenter, OriGenes Technologies Inc., Stratagene, Perkin Elmer, Pharmingen, and Research Genetics. Such vectors may be used for cloning or subcloning nucleic acid molecules of interest and therefore recombinant vectors containing inserts, nucleic acid fragments or genes may also be isolated in accordance with the invention. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, vectors for receiving large inserts (yeast artificial chromosomes (YAC's), bacterial artificial chromosomes (BAC's) and P1 artificial chromosomes (PAC's)) and the like. Other vectors of interest include viral origin vectors (M13 vectors, bacterial phage λ vectors, baculovirus vectors, adenovirus vectors, and retrovirus vectors), high, low and adjustable copy number vectors, vectors which have compatible replicons for use in combination in a single host (e.g., pACYC184 and pBR322) and eukaryotic episomal replication vectors (e.g., pCDM8). The vectors contemplated by the invention include vectors containing inserted or additional nucleic acid fragments or sequences (e.g., recombinant vectors) as well as derivatives or variants of any of the vectors described herein.

Expression vectors useful in accordance with the present invention include chromosomal-, episomal- and virus-derived vectors, e.g., vectors derived from bacterial plasmids or bacteriophages, and vectors derived from combinations thereof, such as cosmids and phagemids, and will preferably include at least one selectable marker (such as a tetracycline or ampicillin resistance genes) and one or more promoters such as the phage lambda PL promoter, and/or the E. coli lac, trp and tac promoters. Other suitable promoters will be known to the skilled artisan.

Among vectors preferred for use in the present invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen Corporation; pGEX, pTrxfus, pTrc99a, pET-5, pET-9, pKK223-3, pKK233-3, pDR540, pRIT5 available from Pharmacia; and pSPORT1, pSPORT2 and pSV-SPORT1, available from Invitrogen Corporation. Other suitable vectors will be readily apparent to the skilled artisan.

Plasmid.

As used herein, the term plasmid means an extrachromosomal genetic element, typically less than about 25 kilobases (kb) in size and more typically about 15 kb to about 2 kb in size.

Isolated. As used herein, the term “isolated” (as in “isolated biological macromolecule”) means that the isolated material, component, or composition has been at least partially purified away from other materials, contaminants, and the like which are not part of the material, component, or composition that has been isolated. For example, an “isolated biological macromolecule” is a macromolecule that has been treated in such a way as to remove at least some of the other macromolecules and cellular components with which it may be associated in the cell, tissue, organ or organism. In particular, the phrases “isolated biological macromolecule,” “isolated nucleic acid molecule” or “isolated vector” refer to macromolecule preparations or vector preparations which contain about 10%, 20%, 30%, 40%, 50%, 55%, 60%, 65%, 70%, 75%, 80%, 85%, 90%, and 93%, preferably more than 95%, 97.5%, and 98%, and most preferably more than 99%, 99.5%, and 99.9% (percentages by weight) of the biological macromolecule of interest. As one of ordinary skill will appreciate, however, a solution comprising an isolated macromolecule may comprise one or more buffer salts and/or a solvents, e.g., water or an organic solvent such as acetone, ethanol, methanol, and the like, and yet the macromolecule may still be considered an “isolated” macromolecule with respect to its starting materials.

Cell disrupting or cell lysing compound or composition. As used herein, “cell disrupting” or “cell lysing” refers to a composition or a component of a composition that effects lysis, rupture, or poration of the cells, tissues, or organisms used as the source of the biological macromolecules to be isolated, such that the macromolecules that are contained in the cell, tissue, or biological source (or portion thereof) are released from the cell, tissue, or organism. According to the invention, the cells, tissues, or organisms need not be completely lysed, ruptured or porated, and all of the macromolecules of interest contained in the source cells, tissues or organisms need not be released therefrom. Preferably, a cell disrupting or cell lysis compound or composition comprises at least 25%, 50%, 75%, 80%, 85%, 90%, 95%, 97%, 99%, or more of the total biological macromolecules of interest, that are contained in the cell, tissue, or organism.

Other terms used in the fields of recombinant DNA technology, biochemistry, protein chemistry and molecular and cell biology as used herein will be generally understood by one of ordinary skill in the applicable arts.

Sources of Biological Macromolecules

The methods, compositions and kits of the invention are suitable for isolation of biological macromolecules from any cellular source, including bacterial cells (particularly Escherichia coli cells), yeast cells, fungal cells, animal cells (particularly insect cells, and mammalian cells including human cells, CHO cells, VERO cells, Bowes melanoma cells, HepG2 cells, and the like), and plant cells, any of which may be transformed cells, established cell lines, cancer cells, or normal cells. Cells may be obtained from cells, tissues, organs or organisms, which may be natural or which may be obtained through any number of commercial sources (including American Type Culture Collection (ATCC), Rockville, Md.; Jackson Laboratories, Bar Harbor, Me.; Cell Systems, Inc., Kirkland, Wash.; Advanced Tissue Sciences, La Jolla, Calif.). Cells that may be used as biological macromolecule sources may be prokaryotic (bacterial, including members of the genera Escherichia (particularly E. coli), Serratia, Salmonella, Staphylococcus, Streptococcus, Clostridium, Chlamydia, Neisseria, Treponema, Mycoplasma, Borrelia, Bordetella, Legionella, Pseudomonas, Mycobacterium, Helicobacter, Agrobacterium, Collectotrichum, Rhizobium, and Streptomyces) or eukaryotic (including fungi or yeasts, plants, protozoans and other parasites, and animals including humans and other mammals). Any virus may also be used as a cellular source of biological macromolecules, particularly nucleic acid molecules, in accordance with the invention. Also suitable for use as sources of biological macromolecules are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus. These cells, tissues and organs may be normal, transformed, or established cell lines, or they may be pathological such as those involved in infectious diseases (caused by bacteria, fungi or yeast, viruses (including AIDS) or parasites), in genetic or biochemical pathologies (e.g., cystic fibrosis, hemophilia, Alzheimer's disease, schizophrenia, muscular dystrophy or multiple sclerosis), or in cancers and cancerous processes. The methods, compositions and kits of the invention are particularly well-suited for isolation of extrachromosomal nucleic acid molecules, including but not limited to MRNA, RNA, tRNA, plasmids, vectors, phagemids, cosmids, cDNA molecules, mitochondrial nucleic acid molecules, and chloroplast nucleic acid molecules, any of which may be single-stranded or double-stranded, linear or circular, supercoiled, and which may be DNA or RNA molecules. In a particularly preferred aspect, the methods of the invention are useful in the isolation of mRNA from eukaryotic cells. Other cells, tissues, viruses, organs and organisms that will be familiar to one of ordinary skill in the art may also be used as sources of biological macromolecules for the preparation of biological macromolecules according to the present invention. Methods

In one aspect, the invention relates to methods for isolating biological macromolecules, particularly nucleic acid molecules such as RNA, MRNA, tRNA, plasmids, vectors, organellar nucleic acid molecules, and the like. Methods according to this aspect of the invention may comprise one or more steps which result in the isolation of one or more biological macromolecules or populations of biological macromolecules (e.g., a library) from the natural environment in which the biological macromolecules are found.

In accordance with the invention, the cells may be lysed or disrupted by contacting them with a composition or compound which causes or aids in cell lysis or disruption, although mechanical or physical forces (e.g., pressure, sonication, temperature (heating, freezing), and/or freeze-thawing etc.) may be used in accordance with the invention. In addition, any combination of mechanical forces, physical forces or lysis compositions/compounds maybe used to disrupt/lyse the cells, so long as the method does not substantially damage the biological macromolecules of interest.

In one preferred embodiment, the cell disrupting or cell lysing compound or composition may comprise one or more detergents, such as sodium dodecylsulfate (SDS), Sarkosyl, Triton X-100, Tween 20, NP-40, Nalkylglucosides, N-alkylmaltosides, glucamides, digitonin, deoxycholate, 3-[(3cholamidopropyl)-dimethylammonio]-1-propane-sulfonate (CHAPS), cetyltrimethyl-ammoniumbromide (CTAB), or Brij 35. The concentration may be about 0.01%-10% (w/v), more preferably about 0.1%-5%, and most preferably about 0.5%. One or more chaeotropic agents such as sodium iodide, sodium perchlorate, guanidine or a salt thereof or urea may be present at a concentration of about 300-1000 mM, more preferably about 500-2000 mM, and most preferably about 1500 mM. One or more enzymes may be present such as lysozyme, lyticase, zymolyase, neuraminidase, Novozym 234, streptolysin, cellulysin, mutanolysin or lysostaphin. Such enzymes may be present at a concentration of about 0.1 to 5 mg/ml. One or more inorganic salts may be present such as sodium chloride, potassium chloride, magnesium chloride, lithium chloride, or praseodymium chloride, at a concentration of about 1 mM to 5M. One or more organic solvents such as toluene, phenol, butanol, isopropyl alcohol, isoamyl alcohol, ethanol, an ether (e.g., diethyl ether, dimethyl ether, or ethylmethyl ether), or chloroform may be present at a concentration of 25 to 60% (v/v). Any other compound which disrupts the integrity of (i.e., lyses or causes the formation of pores in) the membrane and/or cell wall of the cellular source of biological macromolecules (e.g., polymixin B), may be present, or combinations of any of the foregoing. The compositions may also comprise other components, such as chelating agents (e.g., disodium ethylenediaminetetraacetic acid (Na2EDTA), EGTA, CDTA), most preferably at a concentration of about 10 mM. One or more ribonucleases (RNase A, T1, T2, and the like) at concentrations ranging from 1 to 400 μg/ml, proteases (Protinase K, Pronase, pepsin, trypsin, papain, subtilisin) may be present at concentrations ranging from 50 to 1000 μg/ml, or any combination of the foregoing. Desired concentrations and combinations of the active ingredients of the lysis/disruption compositions may be readily determined by those skilled in the art with routine experimentation.

Once the cellular source has been lysed, the lysate, containing the biological macromolecules of interest, is contacted with at least one filter, wherein the pore size of the filters increase in the direction of sample flow. In a preferred embodiment, as the sample flows through the filters, the genomic DNA is sheared by the second filter and the small particles are retained by the first filter. The flow of the biological sample can be facilitated by the use of pressure, vacuum, gravity, centrifugation or combinations thereof. The biological macromolecules of interest which flow through the filter may then be collected.

In one preferred aspect, the methods of the invention comprise:

(a) contacting cells or a cellular source containing the macromolecules of interest with a composition capable of lysing all or substantially all of said cells to give a lysate; and

(b) contacting the lysate with a filter (preferably the filter comprises two or more filters), and wherein the pore size increases in the direction of sample flow; and

(c) promoting the flow of the sample.

Preferably the eluate is collected, e.g. into one or more vials, tubes, microspin tubes, microfuge tubes, spin cartridges, multi-well plates, vials, ampules, bags, and the like.

In an additional embodiment, the invention relates to a process for isolating biological macromolecules from natural sources comprising, separating the lysed natural sources in a sample by filtration, wherein said sample is passed through a filter, the pore size of the filter increasing in the direction of sample flow through the filter. The filter of the invention contains one population of pores that are of sufficient size to trap the flow of cellular debris, and a second population of pores of sufficient size to shear genomic DNA. The pore sizes may range from about 0.1 μm to about 500 μm, and the total thickness of the filter bed may range from about 0.1 mm to about 10 mm. Additionally the filter layers of the filter are composed of sintered polyethylene, polypropylene, polytetrafluorethylene, glass, silica gel, alumina, or packed diatomaceous earth, e.g., cellite or silica gel, interwoven or cemented non-wovens of polypropylene, polyester, glass fibers and-silica, as well as paper, compressed paper, paper non-wovens, hydrophobic polysolfone, hydrophilic polyether sulfone, cellulose, acetylated cellulose, nitrocellulose, polyester, polyolefin, scintered polyethylene, porous ceramics, silica, and polysaccharide. In a preferred embodiment, the sample flow through the filter is promoted by applying positive or negative pressure, or by gravity, or by gravity increased by centrifugation, or by a combination of said measures. The invention also relates to processing several samples simultaneously in devices adapted to microtitration processes.

The invention further relates to a device for isolating biological macromolecules, from natural sources, comprising a hollow body (preferably, but not limited to, cylindrical) having an inlet and an outlet, disposed therein a filter of the invention. Preferably, such filter comprises a multilayered filter bed having increasing pore sizes as seen in the direction of outlet, the filter having a pore size ranging from 0.1 μm to 500 μm, the total thickness of the filter bed being from 0.1 mm to 10 mm, wherein a hydrophobic separating layer is disposed in said cylindrical hollow body.

Compositions

In a related aspect, the invention relates to compositions for use in isolating biological macromolecules of interest.

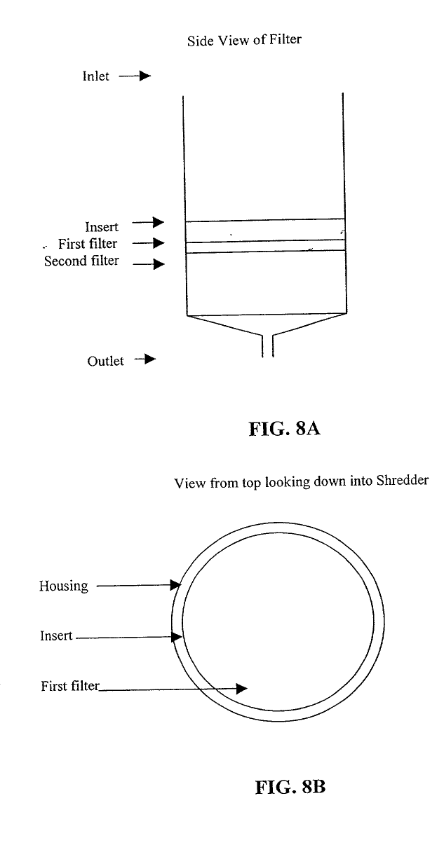

The filter of the invention comprises one or more filter layers. In one preferred embodiment the composition of the invention comprises two filter layers. The first filter layer is capable of retaining fine particles, while the second filter layer, placed downstream of the first filter layer, and having a larger pore size than the first filter layer, is capable of shearing genomic DNA (FIG. 8).

The first filter layer material may include, but is not limited to, hydrophobic polysolfone, hydrophilic polyether sulfone, cellulose, acetylated cellulose, nitrocellulose, polyester, polyolefin, scintered polyethylene, porous ceramics, silica, polysaccharides, and the like. In a preferred embodiment of the invention, the pore size of the first filter layer is sufficient to retard the flow of cellular debris and particles. In a more preferred embodiment the pore size of the first filter layer may range from 0.1-1.0 μm. In a highly preferred embodiment, the filter layer is composed of regenerated cellulose, and has an average pore size of 0.2 μm. In a related aspect, the first filter layer may be prepared in various sizes, shapes, and forms including flat, wafer, cylindrical, rectangular, beads, gels, square, cartridge, swab tip, plug, frit, membrane and the like.

The second filter layer may include any material so long as the pore size is larger than that of the first filter layer, and the second filter layer is capable of shearing genomic DNA. In a preferred embodiment the second filter layer includes, but is not limited to, polyethylene or polypropylene. In a most preferred embodiment of the invention, the pore size of the second filter layer is sufficient to shear genomic DNA molecules. In a highly preferred embodiment, the pore size of the second filter layer may be from 1 μm to >300 μm, most preferably 10-70 μm. In a most preferred embodiment the second filter layer comprises one or more frits, wherein each frit is about 1/16-inch think with an average pore size of 20 μm. In a most preferred embodiment, the second filter layer comprises two frits.

In another preferred embodiment, a filtration apparatus is assembled by placing the second filter into a cartridge housing, placing a first filter on top of the second filter and securing the first and second filters with an insert. Therefore, in a most preferred embodiment the invention relates to a filtration apparatus wherein at least the first and second filters are in a housing adapted to allow said first filter to be contacted with a sample comprising biological macromolecules before said second filter is contacted.

The cartridge housing preferably comprises a hollow, cylindrical or conical body, having an inlet and an outlet, and comprised of a plastic composition, for example polyethylene, polypropylene, polycarbonate, or the like. The cartridge housing is capable of being inserted into a tube, microspin tube, microfuge tube, spin cartridge, multi-well plate, vial, ampule, bag, or the like. Most preferably, the spin cartridge is capable of being inserted into a microspin tube, a microfuge tube, or a multi-well plate typically used in processing of multiple samples, including, for example, 6-well plates, 12-well plates, 24-well plates, 48-well plates, 96-well plates, 384-well plates, and the like, or suitable to fit into other plate sizes such as 35 mm plates, 60 mm plates, 100 mm plates, 150 mm plates, and the like. The inlet should be of sufficient diameter to allow the addition of the first filter, second filter and the insert, and should be sufficiently large to allow addition of a biological sample. The diameter of the outlet is generally smaller than the inlet, and should be of sufficient size to allow elution of the sample into said tube, microspin tube, microfuge tube, spin cartridge, multi-well plate, vial, ampule, bag, and the like.

In accordance with the invention, passage of the cellular contents of interest through the filter may be facilitated by gravity, centrifugation, application of positive or negative pressure, or any combination thereof. The unwanted debris is substantially retained in the first filter, thus allowing the substantial clarification of the lysate. The isolated cellular components of interest may then be further purified by standard techniques and/or further manipulated.

Kits

In another embodiment, the invention relates to kits for use in isolating biological macromolecules of interest, comprising the filter of the invention. Such kits of the invention may comprise one or more components, which may be contained in or include one or more containers such as boxes, cartons, tubes, microspin tubes, microfuge tubes, spin cartridges, multi-well plates, vials, ampules, bags, and the like. In one such aspect, the kits of the invention may comprise one or more compositions from the group consisting of one or more lysing agents, one or more restriction enzymes, one or more polypeptides having nucleic acid polymerase activity (e.g., one or more DNA polymerases which may be thermostable DNA polymerases and/or one or more reverse transcriptases which may be substantially reduced in RNase H activity), one or more cells competent for transformation (e.g., competent cells), transformation reagents, transfection reagents (e.g., cationic lipids), one or more buffers, one or more cells or tissues, one or more biological macromolecules, one or more solvents (organic or inorganic), one or more alcohols, or other components or reagents that may be useful in conjunction with further purification, processing and analysis of the isolated macromolecules of the invention.

In one such kit, the kit comprises the filtration apparatus of the invention, one or more cell lysis/disrupting compositions and compounds as well as elution and wash compositions for use in the methods and compositions of the present invention.

In accordance with the invention the elution and wash compositions may include, but are not limited to water, buffered aqueous solutions, buffered aqueous salt solutions, alcohols, alcohol solutions, other organic solvents, and the like.

The kits of the invention may further comprise one or more additional components or reagents that may be useful in further processing, analysis, or use of the biological macromolecules isolated or purified according to the invention, for example components or reagents useful in nucleic acid amplification, hybridization, labeling, quantization, sequencing, cloning, transfection, transcription, translation, and the like. Such reagents or components may, for example, include one or more restriction enzymes, one or more polypeptides having reverse transcriptase activity, one or more polypeptides having nucleic acid polymerase activity, one or more cells competent for transformation, one or more transfection reagents (e.g., lipids) and other reagents that will be familiar to one of ordinary skill in the art.

Polypeptides having reverse transcriptase activity for use in the kits of the invention may include any polypeptide having the ability to synthesize a DNA molecule from an RNA template molecule. In one embodiment, the polypeptides having reverse transcriptase activity may be substantially reduced in RNase H activity. Suitable polypeptides having reverse transcriptase activity for use in the kits of the invention include, but are not limited to, M-MLV reverse transcriptase, RSV reverse transcriptase, AMV reverse transcriptase, RAV reverse transcriptase, MAV reverse transcriptase or HIV reverse transcriptase. These polypeptides having reverse transcriptase activity may be substantially reduced in RNase H activity; preferred such polypeptides include M-MLV H⁻ reverse transcriptase, RSV H⁻ reverse transcriptase, AMV H⁻ reverse transcriptase, RAV H⁻ reverse transcriptase, MAV H⁻ reverse transcriptase and HIV H⁻ reverse transcriptase. Methods for the production and use of such polypeptides having reverse transcriptase activity, including those which are substantially reduced in RNase H activity, are described in detail in commonly owned, PCT Application WO 98/47912, published Oct. 29, 1998, the disclosure of which is incorporated herein in its entirety.

Polypeptides having nucleic acid polymerase activity for use in the kits of the invention may be any polypeptide that can synthesize a nucleic acid molecule from a nucleic acid template, typically in the 5′ to 3′ direction. The nucleic acid polymerases used in the kits of the present invention may be mesophilic or thermophilic, and are preferably thermophilic. Preferred mesophilic DNA polymerases include T7 DNA polymerase, T5 DNA polymerase, Klenow fragment DNA polymerase, DNA polymerase III and the like. Preferred thermostable DNA polymerases that may be used in the kits of the invention include Taq, Tne, Tma, Pfu, Tfl, Tth, Stoffel fragment, VENT™ and DEEPVENT™ DNA polymerases, and mutants, variants and derivatives thereof (U.S. Pat. No. 5,436,149; U.S. Pat. No. 4,889,818; U.S. Pat. No. 4,965,188; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); Flaman, J. -M, et al., Nucl. Acids Res. 22(15):3259-3260 (1994)). For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; and U.S. Pat. No. 5,512,462; Barnes, W. M., Gene 112:29-35 (1992), the disclosures of which are incorporated herein in their entireties. Examples of DNA polymerases substantially lacking in 3′ exonuclease activity include, but are not limited to, Taq, Tne(exo⁻), Tma(exo⁻), Pfu(exo⁻), Pwo(exo⁻) and Tth DNA polymerases, and mutants, variants and derivatives thereof. RNA polymerases such as T3, T5 and SP6 and mutants, variants and derivatives thereof may also be used in accordance with the invention.

Isolated Nucleic Acid Molecules, Vectors, and Host Cells

The invention also relates to isolated biological macromolecules that are prepared according to the methods of the invention. Preferred biological macromolecules that may be isolated according to the present invention include, but are not limited to, RNA, mRNA, tRNA, plasmids, large molecular weight plasmids (BAC's, PAC's and YAC's), vectors, cDNA molecules or libraries, cosmids, phagemids, organellar nucleic acid molecules (e.g., those isolated from organelles such as mitochondria or chloroplasts), proteins, peptides and the like. The nucleic acid molecules may be single stranded or double stranded, circular or linear, supercoiled, and may be comprised of DNA, RNA, or a combination of DNA and RNA. In one preferred embodiment, the isolated nucleic acid molecules of the invention are mRNA molecules, particularly those isolated, for example, from bacterial cells.

The invention also provides recombinant host cells comprising the isolated biological macromolecules of the invention. Representative host cells (prokaryotic or eukaryotic) that may be produced according to the invention include, but are not limited to, bacterial cells, yeast cells, plant cells and animal cells. Such suitable host cells are available commercially, for example from Invitrogen Corporation, ATCC (Manassas, Va.), and other commercial sources that will be familiar to one of ordinary skill in the art. Host cells comprising the vectors, recombinant vectors or isolated nucleic acid molecules of the invention may be prepared by inserting the isolated nucleic acid molecules or vectors of the invention into the host cells, using well-known transformation, electroporation or transfection techniques that will be familiar to one of ordinary skill in the art. According to this aspect of the invention, introduction of isolated nucleic acid molecules into a host cell to produce a host cell comprising the nucleic acid molecules can be effected by any known method of introducing nucleic acid molecules into host cells, including but not limited to calcium phosphate transfection, DEAE-dextran mediated transfection, cationic lipid-mediated transfection, electroporation, transduction, transformation (e.g., of competent cells particularly E. coli cells), infection or other methods. Such methods are described in many standard laboratory manuals, such as Davis et al., “Basic Methods In Molecular Biology” (1986) and Maniatis et al., “Molecular Cloning: A Laboratory Manual,” Cold Spring Harbor Laboratory, Cold Spring Harbor, N.Y. (1982). Appropriate culture media and cultivation conditions for the transformed or transfected host cells are known in the art.

In addition, the invention provides methods for producing a recombinant polypeptide encoded by an isolated nucleic acid molecule of the invention, and polypeptides produced by these methods. According to this aspect of the invention, a recombinant polypeptide may be produced by culturing any of the above recombinant host cells comprising the isolated nucleic acid molecules, recombinant vectors or vectors of the invention, under conditions favoring production of a polypeptide therefrom, and isolation of the polypeptide. Methods for culturing recombinant host cells, and for production and isolation of polypeptides therefrom, are well-known to one of ordinary skill in the art.

Uses of Isolated Nucleic Acid Molecules

The nucleic acid molecules that are isolated by the compositions, methods and kits of the present invention may be further characterized or manipulated, for example by cloning, sequencing, amplification, labeling, nucleic acid synthesis, endonuclease digestion and the like.

The isolated nucleic acid molecules of the invention may be used in methods for amplifying and sequencing nucleic acid molecules. Amplification methods which may be used in accordance with the present invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822). The isolated nucleic acid molecules may also be used in complex PCR-based nucleic acid fingerprinting techniques such as Random Amplified Polymorphic DNA (RAPD) analysis (Williams, J. G. K., et al., Nucl. Acids Res. 18(22):6531-6535, 1990), Arbitrarily Primed PCR (AP-PCR; Welsh, J., and McClelland, M., Nucl. Acids Res. 18(24):7213-7218,1990), DNA Amplification Fingerprinting (DAF; Caetano-Anollés et al., Bio/Technology 9:553-557, 1991), microsatellite PCR or Directed Amplification of Minisatellite-region DNA (DAMD; Heath, D. D., et al., Nucl. Acids Res. 21(24): 5782-5785, 1993), and Amplification Fragment Length Polymorphism (AFLP) analysis (EP 0 534 858; Vos, P., et al., Nucl. Acids Res. 23(21):4407-4414, 1995; Lin, J. J., and Kuo, J., FOCUS 17(2):66-70,1995). In a particularly preferred aspects, the invention may be used in methods of amplifying or sequencing a nucleic acid molecule comprising one or more polymerase chain reactions (PCRs), such as any of the PCR-based methods described above. Nucleic acid sequencing methods according to this aspect of the invention may comprise both cycle sequencing (sequencing in combination with linear amplification) and standard sequencing reactions, according to methods that are well-known in the art. In another particularly preferred embodiment, the invention may be used in methods for making one or more cDNA molecules, populations of cDNA molecules or libraries of cDNA molecules. The cDNA molecules may be single stranded or double stranded.

Alternatively, nucleic acid molecules isolated according to the present invention may be used for the manufacture of various materials in industrial processes by methods that are well-known in the art. Such materials include, but are not limited to, hybridization probes, therapeutic proteins (dependent upon transcription and translation of the isolated nucleic acid molecules, or the production of synthetic peptides or proteins with amino acid sequences deduced from the nucleotide sequences of the specific nucleic acid molecules), gene therapy vehicles and compositions, molecular weight markers, and the like.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Animal or Plant Cells

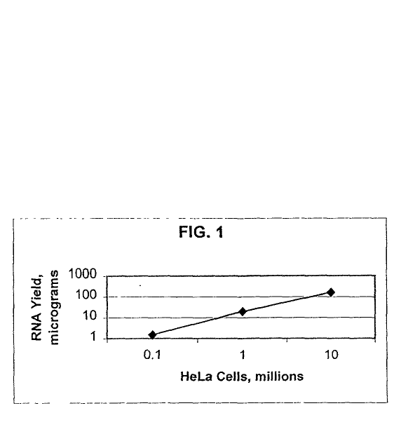

HeLa cells (up to 1×10⁷ cells) were disrupted in 0.6 ml of guanidinium isothiocyanate lysis buffer (4 M guanidinium isothiocyanate, 50 mM Tris HCl, pH 7.5, 25 mM EDTA), transferred to the filter of the invention. This embodiment of the invention comprises a first regenerated cellulose layer, 6.9 mm in diameter, with a pore size of 0.2 μm; and a second high density polyethylene layer, 7.3 mm in diameter and ⅛ inch thick (comprising two 1/16 inch thick frits), with a 20 μm pore size. The filter is contained in a 3 cm long conical housing, 1.3 cm in diameter at the top, tapering to 0.4 cm at the bottom. This housing was then placed in a 2-ml conical centrifuge tube, and centrifuged for two minutes. The filter of the invention was removed, and an equal volume of 70% ethanol was added to the flow-through. The sample was mixed and transferred to a glass fiber RNA-binding cartridge contained in a 2-ml tube. The sample was centrifuged, the RNA-binding cartridge was washed several times. The RNA was eluted from the cartridge with water. (FIG. 1)

### Example 2

### Animal Tissues

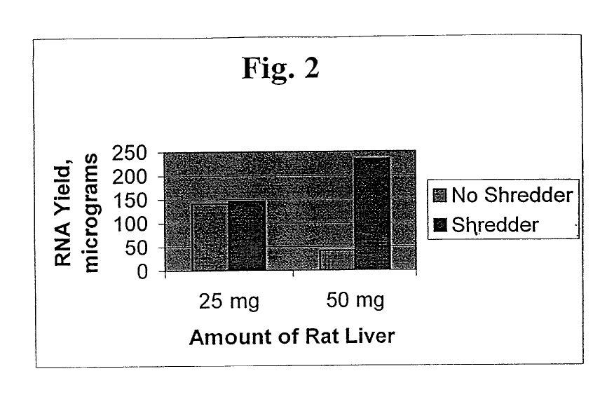

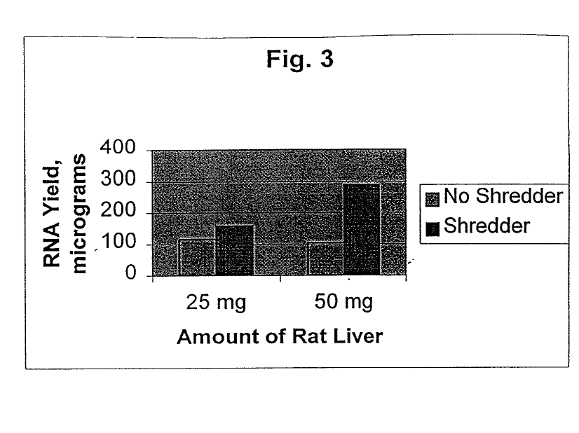

Rat liver, brain or spleen (1 mg to 60 mg) was disrupted in 0.3-0.6 ml of guanidinium isothiocyanate lysis buffer, transferred to the filter of the invention (as described in Example 1), contained in a 2-ml conical centrifuge tube, and centrifuged for two minutes. The filter was removed, and an equal volume of 70% ethanol was added to the flow-through. The sample was mixed and transferred to a glass fiber RNA-binding cartridge contained in a 2-ml tube. The sample was centrifuged, the RNA-binding cartridge was washed several times. The RNA was eluted from the cartridge with water. As shown in FIGS. 2 and 3, the RNA yield when the composition of the invention is used is significantly higher than when the composition of the invention is not used. This is particularly true when RNA is isolated from larger amounts of tissue (50 mg).

### Example 3

### Animal Tissues

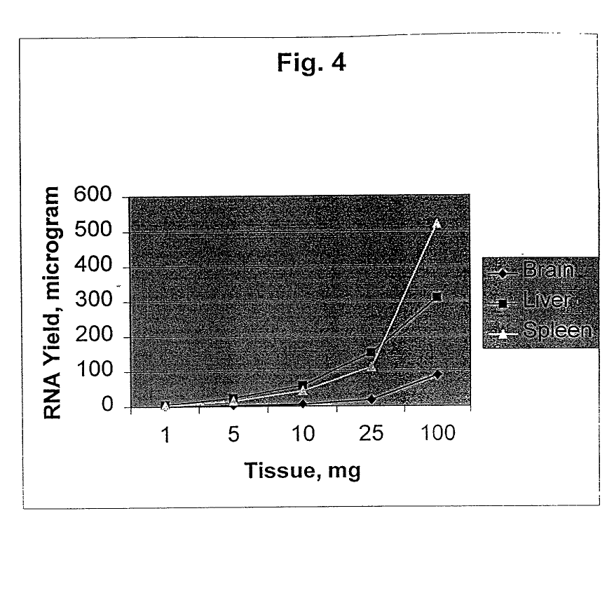

Rat liver, brain or spleen (>60 mg to 100 mg) was disrupted in 1.2 ml of guanidinium isothiocyanate lysis buffer. The lysate was divided into aliquots and each aliquot was transferred to the filter of the invention (as described in Example 1), contained in a 2-ml conical centrifuge tube, and centrifuged for two minutes. The flow-through was re-applied to the filter and centrifuged for 2 minutes. The filters of the invention were removed, the two aliquots were combined in a 15-ml conical tube, and an equal volume of 70% ethanol was added, the sample was vortexed for 2 minutes at high speed, and aliquots were processed on an RNA-binding cartridge contained in a 2-ml tube. The RNA-binding cartridge was washed several times and the RNA was eluted from the cartridge with water. (FIG. 4)

### Example 4

### Plant Tissues

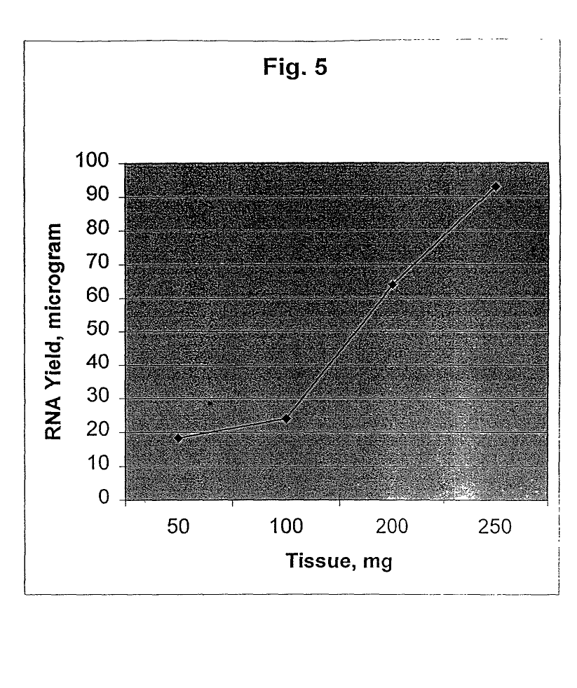

Frozen, ground plant leaves (50 mg to 100 mg) were disrupted in 0.5 ml of guanidinium isothiocyanate lysis buffer, transferred to the filter of the invention (as described in Example 1) contained in a 2-ml conical centrifuge tube, and centrifuged for two minutes. The filter was removed, and 0.5 volume of 100% ethanol was added to the flow-through. The sample was mixed and transferred to a glass fiber RNA-binding cartridge contained in a 2-ml tube. The sample was centrifuged, the RNA-binding cartridge was washed several times and the RNA was eluted from the cartridge with water. (FIG. 5).

### Example 5

### Human Whole Blood

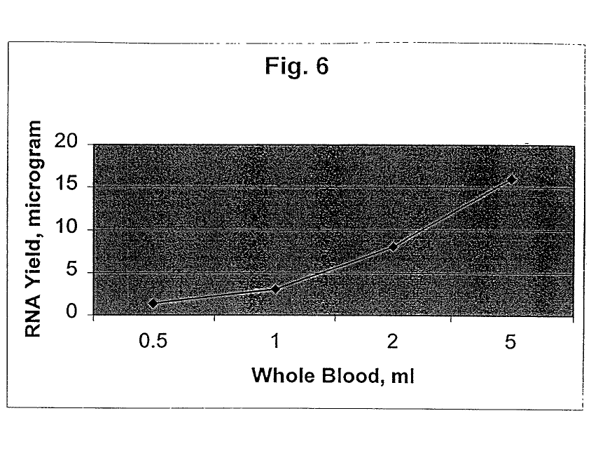

Human whole blood (0.2-5 ml) was treated with red blood cell-selective lysis buffer, centrifuged to collect the white blood cells, and washed one time with the same reagent. The white blood cells were disrupted in guanidinium isothiocyanate lysis buffer, transferred to the filter of the invention (as described in Example 1) contained in a 2-ml conical centrifuge tube, and centrifuged for two minutes. The filter was removed, and an equal volume of 70% ethanol was added to the flow-through. The sample was mixed and transferred to a glass fiber RNA-binding cartridge contained in a 2-ml tube. The sample was centrifuged, the RNA-binding cartridge was washed several times and the RNA was eluted from the cartridge with water. (FIG. 6).

### Example 6

### Yeast Cells

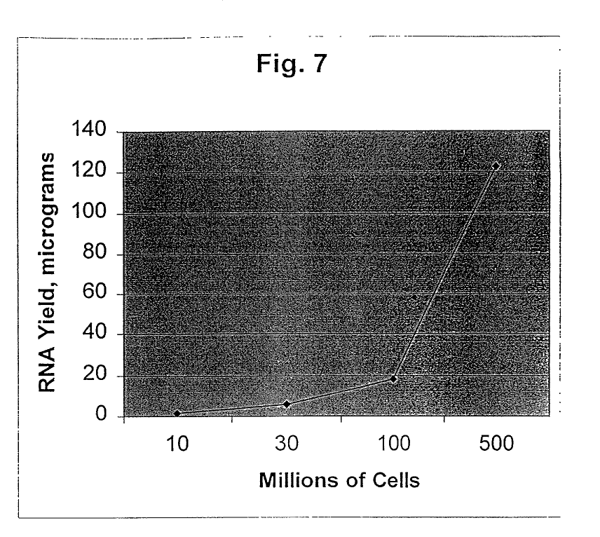

Up to 5×10⁸ S. Cerevisiae yeast cells from an overnight growth were suspended in 500 ml guanidinium isothiocyanate lysis buffer. The suspension was added dropwise onto powdered dry ice in a porcelain mortar and crushed with a pestle until the dry ice had evaporated and the and the residual paste had melted. The liquid was transferred to the filter of the invention (as described in Example 1), contained in a 2-ml conical centrifuge tube and centrifuged at 12,000× g for 2 minutes. An equal volume of 70% ethanol was added to the homogenate, mixed by pipetting up and down, and passed through a glass fiber RNA binding cartridge. The sample was centrifuged, the RNA-binding cartridge was washed several times and the RNA was eluted with water. (FIG. 7)

Results

As the viscosity of the lysate increases, greater differences in RNA yields are observed between lysate homogenized with the filter of the invention and lysate not homogenized. FIG. 1 shows that there is greater discrepancy in RNA yields between lysate and homogenized lysate at 50 mg compared to 25 mg of rat liver lysed in 0.6 ml of guanidine isothiocyanate solution and ethanol precipitated. FIG. 2 shows that a similar effect is observed if the lysate and homogenized lysate are processed over RNA binding cartridges.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method for isolation of biological macromolecules, said method comprising contacting at least one filter with a biological sample comprising the biological macromolecules of interest, wherein the pore size of said filter increases in the direction of sample flow.

2. The method of claim 1, wherein said biological sample is a cellular lysate.

3. The method of claim 2, wherein said cellular lysate is derived from eukaryotic cells.

4. The method of claim 2, wherein said cellular lysate is derived from prokaryotic cells.

5. The method of claim 3, wherein said eukaryotic cells are selected from the group consisting of fungi, fish cells, yeast cells, plant cells and animal cells.

6. The method of claim 1, wherein said biological macromolecules are nucleic acid molecules.

7. The method of claim 1, wherein said biological macromolecules are protein molecules.

8. The method of claim 6, wherein said nucleic acid molecules are RNA molecules.

9. The method of claim 8, wherein said RNA molecules are mRNA molecules.

10. The method of claim 6, wherein said nucleic acid molecules are DNA molecules.

11. The method of claim 10, wherein said DNA molecules are vectors or plasmids.

12. The method of claim 1, wherein said filter comprises at least two filter layers.

13. The method of claim 12, wherein a first filter has a pore size smaller than the second filter, and wherein said sample first contacts said first filter layer and then contacts said second filter layer.

14. The method of claim 13, wherein said second filter layer comprises at least one frit.

15. The method of claim 14, wherein said second filter layer comprises pores of sufficient size to shear genomic DNA, and said pore size is larger than that of the first filter layer.

16. The method of claim 15, wherein said pore size of said second filter layer is about 1 μm to 500 μm.

17. The method of claim 16, wherein said pore size of said second filter layer is about 10 μm to 70 μm.

18. The method of claim 17, wherein said pore size of said second filter layer is about 20 μm.

19. The method of claim 14, wherein said second filter layer comprises two frits.

20. The method of claim 19, wherein each of said frits are about 1/16 inch thick.

21. The method of claim 13, wherein said first filter layer comprises pores of sufficient size to retard the flow of cellular debris and particles.

22. The method of claim 21, wherein said pores of said first filter layer are about 0.1 μm to 1.0 μm in diameter.

23. The method of claim 21, wherein said pores of said first filter layer are about 0.2 μm in diameter.

24. The method of claim 13, wherein said second filter layer is comprised of polyethylene, polypropylene or a combination thereof.

25. The method of claim 13, wherein said first filter layer is comprised of one or more materials selected from the group consisting of hydrophobic polysolfone, hydrophilic polyether sulfone, cellulose, acetylated cellulose, nitrocellulose, polyester, polyolefin, scintered polyethylene, porous ceramics, silica, and polysaccharide.

26. The method of claim 25, wherein said first filter layer is comprised of regenerated cellulose.

27. The method of claim 26, wherein said first filter layer is comprised of regenerated cellulose, with a pore size of about 0.2 μm, and comprised of polyethylene or polypropylene, with an average pore size of about 20 μm.

28. The method of claim 1, wherein said filter is provided in a form selected from the group consisting of wafer, cylindrical, rectangular, beads, gels, square, cartridge, swab tip, plug, frit, membrane, sheets or inserts.

29. The method of claim 1, wherein said filter is provided in a form that is suitable to be inserted into a tube, microspin tube, microfuge tube, spin cartridge, vial, ampule, bag or suitable to fit multi-well plates typically used in processing of multiple samples, including, 6-well plates, 12-well plates, 24-well plates, 48-well plates, 96-well plates, 384-well plates, and the like, or suitable to fit into other plate sizes such as 35 mm plates, 60 mm plates, 100 mm plates, 150 mm plates, and the like.

30. The method of claim 1, wherein the flow of the sample is facilitated by centrifugation, gravity, pressure, vacuum, or any combination thereof.

31. A method for isolation of biological macromolecules, said method comprising;
(a) contacting cells or cellular source containing the macromolecules of interest with a composition capable of lysing all or substantially all of said cells to give a lysate; and
(b) contacting the lysate with a filter, wherein the filter comprises two or more filters, and wherein the pore size increases in the direction of sample flow; and
(c) promoting the flow of the sample through the filter.

32. An apparatus for use in isolating biological macromolecules, comprising one or more filters, wherein the pore size of said filters increases in the direction of sample flow.

33. The apparatus of claim 32, further comprising at least a first and second populations of pores.

34. The apparatus of claim 33, wherein said first population comprises pores of sufficient size to retard the flow of cellular debris, and said second population, which is downstream of said first population, comprises pores of sufficient size to shear genomic DNA.

35. The apparatus of claim 32, wherein a first and second filter are contained in a cartridge housing adapted to allow said first filter to be contacted with a sample comprising biological macromolecules before said second filter is contacted.

36. The apparatus of claim 35, wherein said second filter is a frit.

37. The apparatus of claim 36, wherein said second filter comprises pores of sufficient size to shear genomic DNA.

38. The apparatus of claim 37, wherein said pore size of said second filter is about 1 μm to 500 μm.

39. The apparatus of claim 38, wherein said pore size of said second filter is about 10 μm to 70 μm.

40. The apparatus of claim 39, wherein said pore size of said second filter is about 20 μm in diameter.

41. The apparatus of claim 37, wherein said first filter comprises pores of sufficient size to retard the flow of cellular debris and particles.

42. The apparatus of claim 41, wherein said pore size of said first filter is about 0.1 μm to 1.0 μm.

43. The apparatus of claim 42, wherein said pore size of said first filter is about 0.2 μm.

44. The apparatus of claim 36, wherein said frit is comprised of polyethylene, polypropylene or a combination thereof.

45. The apparatus of claim 35, wherein said first filter is comprised of one or more materials selected from the group consisting of hydrophobic polysolfone, hydrophilic polyether sulfone, cellulose, acetylated cellulose, nitrocellulose, polyester, polyolefin, scintered polyethylene, porous ceramics, silica, and polysaccharide.

46. The apparatus of claim 45, wherein said first filter is comprised of regenerated cellulose.

47. The apparatus of claim 46, wherein said first filter is comprised of regenerated cellulose, with an average pore size of 0.2 μm, and said frit is comprised of polyethylene or polypropylene, with an average pore size of 20 μm.

48. The apparatus of claim 35, wherein said cartridge housing preferably comprises a hollow, cylindrical or conical body, having an inlet and an outlet.

49. The apparatus of claim 48, wherein said cartridge housing is capable of being inserted into a tube, microspin tube, microfuge tube, spin cartridge, multi-well plate, vial, ampule or bag.

50. An apparatus for use in isolating biological macromolecules, comprising at least a first and second filters, wherein;
(a) said first filter comprises regenerated cellulose, with an average pore size of about 0.2 μm; and
(b) said second filter is comprised of polyethylene or polypropylene, with an average pore size of about 20 μm; and
(c) the second filter is placed in a cartridge housing; and
(d) the first filter is placed on top of the second filter; and
(e) said filters are secured to said cartridge housing with an insert.

51. A kit for use in isolating a nucleic acid molecule or a population of nucleic acid molecules, said kit comprising the apparatus of claim 32.

52. The kit of claim 51 of the invention, further comprising one or more additional compositions or reagents for use in further characterization or manipulation of the isolated biological macromolecule of the invention and a lysis composition.

53. The kit of claim 52, wherein said lysis composition is selected from the group consisting of sodium dodecylsulfate, Sarkosyl, Triton X-100, Tween 20, NP-40, N-alkylglucosides, N-alkylmaltosides, glucamides, digitonin, deoxycholate, 3-[(3-cholamidopropyl)-dimethylammonio]-1-propane-sulfonate (CHAPS), cetyltrimethyl-ammoniumbromide (CTAB), Brij 35, sodium iodide, sodium perchlorate, guanidine, guanidine salts, urea, lysozyme, lyticase, zymolyase, neuraminidase, Novozym 234, streptolysin, cellulysin, mutanolysin, lysostaphin, sodium chloride, potassium chloride, magnesium chloride, lithium chloride, praseodymium, toluene, phenol, butanol, isopropyl alcohol, isoamyl alcohol, ethanol, diethyl ether, dimethyl ether, ethylmethyl ether, and chloroform, or any combination thereof.

54. The kit of claim 52 of the invention, wherein said additional compositions are selected from the group consisting of restriction enzymes, polypeptides having nucleic acid polymerase activity, cells competent for transformation, other transformation reagents, transfection reagents or other components or reagents that may be useful in conjunction with further purification, processing and analysis of the isolated macromolecules of the invention.

55. A process for isolating biological macromolecules comprising, separating a lysed natural source in a sample by filtration, wherein said sample is passed through a filter, the pore size of said filter increasing in the direction of sample flow through the filter.

56. The process according to claim 55, wherein the sample flow through the filter is promoted by applying positive or negative pressure, or by gravity, or by gravity increased by centrifugation, or by a combination thereof.

57. The process according to claim 55, wherein said nucleic acid is plasmid DNA or genomic DNA having a size of from 1 to 50 kb (kilo base pairs).

58. The process according to claim 55, wherein said sample is passed through a filter composed of a multitude of layers wherein, with respect to a particular initial pore size, the subsequent layers have increasingly larger pore sizes.

59. The process according to claim 55, wherein said sample is passed through a filter comprising at least one layer whose pore size increases in the direction of sample flow.

60. The process according to claim 55, wherein said pore size ranges from 1 μm to 500 μm, the total thickness of the filter bed being from 0.1 mm to 10 mm.

61. The process according to claim 55, wherein said sample is passed through a two-layered filter bed wherein the first filter layer has a pore size of from 0.1 to 1.0 μm, and the second filter layer has a pore size of from 1 to 500 μm.

62. The process according to claim 55, wherein said filter layers of said filter are composed of sintered polyethylene, polypropylene, polytetrafluorethylene, glass, silica gel, alumina, or packed diatomaceous earth, e.g., cellite or silica gel, interwoven or cemented non-wovens of polypropylene, polyester, glass fibers and-silica, as well as paper, compressed paper, paper non-wovens, hydrophobic polysolfone, hydrophilic polyether sulfone, cellulose, acetylated cellulose, nitrocellulose, polyester, polyolefin, scintered polyethylene, porous ceramics, silica, and polysaccharide.

63. The process according to claim 55, wherein several samples are processed simultaneously.

64. A device for performing the process according to claim 55, comprising a cylindrical hollow body having an inlet and an outlet, disposed therein a filter comprising increasing pore sizes as seen in the direction of outlet, the filter having a pore size ranging from 0.1 μm to 500 μm, the total thickness of the filter bed being from 0.1 mm to 10 mm.

65. The device according to claim 64, wherein a hydrophobic separating layer is disposed in said cylindrical hollow body.
